(19) United States
(12) Patent Application Publication
(10) Pub. No.: US 2018/0148616 A1
(43) Pub. Date: May 31, 2018

(54) HOT MELT ADHESIVES FOR BONDING ELASTOMERIC COMPONENTS, NONWOVEN MATERIALS, AND THERMOPLASTIC FILMS

(71) Applicant: Bostik, Inc., Wauwatosa, WI (US)

(72) Inventors: Genta OKAZAKI, Tenri-Shi (JP); Kimberly E. SECRIST, Wauwatosa, WI (US); Steven D. GRAY, Mequon, WI (US)

(21) Appl. No.: 15/822,790

(22) Filed: Nov. 27, 2017

Related U.S. Application Data
(60) Provisional application No. 62/426,774, filed on Nov. 28, 2016.
(60) Provisional application No. 62/527,444, filed on Jun. 30, 2017.

(51) Int. Cl. C09J 123/12; C09J 123/14; C09J 123/08; C09J 5/06; C09J 11/04; C09J 11/06; C09J 11/08

(52) U.S. Cl. CPC C09J 123/12; C09J 123/14; C09J 123/08; C09J 5/06; C09J 11/04; A61F 13/5611; C09J 11/08; C09J 2423/00; C09J 2425/00; C09J 2431/00; C09J 2491/00; C09J 11/06

(57) ABSTRACT

A hot melt adhesive composition comprises a polymer blend based on a first polymer component having a low melting point and is selected from a polypropylene homopolymer and a copolymer of propylene and ethylene and mixtures thereof; a second polymer component comprising an amorphous polyolefin; and about 30% to about 75% by weight of a tackifying resin. The composition optionally further contains a plasticizer, an antioxidant, a wax, a filler, a colorant, a UV absorber, another polymer, or combinations thereof. The hot melt composition has a viscosity equal to or less than about 80,000 cP at 180° C. and is useful for a variety of industrial applications including bonding together the substrates used in disposable hygiene products, such as nonwoven layers, elastic attachments, and thermoplastic films (polyolefin, polylactic acid, etc.). The hot melt adhesive composition may be dually functional, serving as an elastic component adhesive and a construction adhesive.

### CROSS-REFERENCE TO RELATED APPLICATIONS

This application claims the benefit of U.S. Patent Application Nos. 62/426,774, filed on Nov. 28, 2016, and 62/527,444, filed on Jun. 30, 2017.

### FIELD OF THE INVENTION

The present invention relates to hot melt adhesives, and more particularly to hot melt adhesives made from blends of low melting point polypropylene-based polymers or copolymers and amorphous poly-alpha olefins (APAO). These adhesives are useful in bonding elastomeric components to various substrates and are useful as constructive adhesives for the manufacture of disposable consumer articles, such as diapers, feminine sanitary napkins, adult incontinent products, medical gowns, and the like.

### BACKGROUND OF THE INVENTION

Hot melt adhesives are used to form bonds between various substrates for a wide range of commercial end-uses. For example, hot melt adhesives are employed to bond nonwoven materials, polymeric films, and elastomeric components in numerous fabricated articles. In such applications, the hot melt adhesive is used to bond elastomeric components such as strands, films, attachment tabs or panels, and other continuous or discrete forms between fabrics, synthetic fabrics, nonwoven materials, and various polymeric films. For example, adhesively bonded elastic strands are used to improve the fit of disposable hygiene products around the leg and waist areas of the article. These applications require the adhesive to form strong bonds to the substrates without compromising the elasticity of the strand needed for the garment to comfortably adapt to the wearer's movement while maintaining a reliable seal to retain fluids. Hot melt adhesives used to attach elastic components to at least one other substrate are referred to herein as “elastic component adhesives.”

Hot melt “construction adhesives” for disposable consumer articles bond various nonwoven materials with low surface energy thermoplastic films, such as polylactic acid, polyethylene, or untreated polypropylene. The use of thinner polyolefin back sheets in the manufacture of disposable articles requires the use of lower viscosity hot melts in order to prevent burn-through and distortion when the adhesive is applied. Construction adhesives should have good shear strength, but additionally must have strong peel strength (particularly at low add-on levels, such as 1 or 2 grams per square meter). On the other hand, elastic component adhesives must demonstrate good creep resistance.

Hot melt adhesives can be applied using a wide range of application methods and process conditions. Hot melt adhesives can be sprayed or coated as thin filaments or layers of various patterns to substrates or to elastic components which are then affixed to various materials. Once cooled, the adhesive needs to fulfill multiple requirements such as displaying suitable bond strength as measured by peel force or bond retention during and/or after mechanical stress. In certain applications, bonding performance must be maintained during and after mechanical stress has been applied to articles which have undergone long term or thermally accelerated aging.

Hot melt adhesives can be based on polymers such as polyolefins (ethylene- or propylene-based polymers and the like), or functionalized polyolefins (ethylene or propylene copolymers and the like produced with oxygen and other heteroatom containing monomers), or styrenic block copolymers containing at least one rubbery phase, such as poly(styrene-b-isoprene-b-styrene) (SIS) or poly(styrene-b-butadiene-b-styrene) (SBS). When SIS, SBS, and similar block copolymers are employed, the styrenic phase is generally thought to provide cohesive strength while the poly(diene) phase is believed to impart the elastomeric behavior critical to performance of fabricated components that must withstand mechanical forces in an end use application such as elastic component adhesives.

Over the years, many different olefinic polymers have been used in the formulation of hot melt adhesives. The first of these were amorphous polypropylenes (APP) that are characterized by having a random steric orientation of the pendant methyl group along the carbon backbone of the polymer chain. The lack of stereoregularity frustrates the development of crystallinity of APP systems, making them compatible with the various tackifiers, plasticizers, waxes, and fillers used to tailor the overall performance of the adhesive.

Later, other olefin polymers became available that offered improved properties over the original amorphous polypropylene polymers. These are referred to as amorphous poly-alpha olefins (APAOs). APAOs are made using a variety of monomers, including but not limited to, propylene, ethylene, and butene. They are typically random polymers that possess fairly broad molecular weight distributions (polydispersity index >3.0) and can be produced employing a variety of Ziegler-Natta catalyst systems.

Typically, hot melt adhesives based solely on low crystallinity APP or APAO materials, however, fail to meet the bond retention performance criteria for elastic applications as they generally yield at low mechanical forces, have poor elasticity, and cannot maintain strong bonding in articles that have undergone long term or thermally accelerated aging. Additionally, formulations containing only very low crystallinity polyolefins tend to develop properties slowly. The latter issue can be troublesome when employed on porous substrates such as nonwovens commonly used in hygiene applications where slow set-up can lead to adhesive over-penetration, compromising performance of the final laminate and, in extreme cases, cause build-up of the adhesive on process equipment and potentially blocking.

More recently, metallocene and other single site catalysis (SSC) have been developed to produce polyolefins with more precisely tailored properties which overcome some of these limitations. For example, the molecular weight distribution can be controlled using catalysts of these types to provide polymers with significantly narrower polydispersity values compared to those produced employing traditional Ziegler-Natta catalysts. The narrow polydispersities of these materials allows low viscosity adhesives to be produced that do not contain extremely short polymer chains which can compromise physical properties. Single-site catalysts also are capable of incorporating far greater levels of comonomer compared to Ziegler-Natta catalysts. This allows high levels of comonomers, such as 1-butene, 1-hexene, and 1-octene, to be incorporated into ethylene-based polymers to provide medium to low density polyethylene copolymers that can be made into high clarity films with excellent mechanical properties. Examples of ethylene-based copolymers of this class include Affinity® and Engage® polymers from the Dow Chemical Company. Similarly, single-site catalysts have been developed which allow propylene based copolymers to be produced that contain high levels of ethylene and/or other alpha-olefins. Examples of propylene based copolymer systems include Vistamaxx® polymers from ExxonMobil and Versify® grades available from the Dow Chemical Company.

Single-site catalysts can be further exploited to control the chain architecture of polyolefins and their copolymers. These catalysts govern the degree of stereo- and regio-defects along the polymer chains and, in turn, the overall crystallinity and final properties. Control of polymer stereo-regularity using these catalysts can be performed such that pendant substituents of neighboring backbone carbons (“diads”) are primarily arrayed in an identical (“meso”) fashion to provide highly isotactic polymers. Conversely, single-site catalysts can be designed such that side-branch alkyl groups are oriented in an opposing (“racemic”) fashion to afford syndiotactic polymers. Materials with highly controlled tacticity that contain very low levels of stereo-errors (less than 0.50 mol %), such as isotactic and syndiotactic polypropylene homopolymers, are generally stiff, high melting materials.

Recently, catalysts have been developed that target a fixed level of stereo-defects to allow for fine control of polymer properties. Using catalysts designed to selectively introduce a controlled level of stereo-errors can provide materials that, while compositionally identical to other polypropylene homopolymers, display enhanced flexibility and are lower melting. Examples of this class of polymers include L-MODU S400, S600, and S901 propylene-based homopolymers available from Idemitsu Chemicals. While these polymers have been used to make hot melt adhesives with better adhesion characteristics, they have not been widely used in applications requiring the formation of strong initial bonds to a variety of substrates including elastic materials that must be rigorously maintained with long-term aging under various thermal conditions. Moreover, it would be advantageous to provide a single adhesive which performs well as both a construction adhesive and an elastic component adhesive (i.e., are “dually functional”).

### SUMMARY OF THE INVENTION

In view of its purposes, an embodiment of the present invention provides a hot melt adhesive composition comprising a first polymer component having a low melting point and selected from the group consisting of a polypropylene homopolymer and a copolymer of propylene and ethylene and mixtures thereof; a second polymer component comprising an amorphous polyolefin; and a tackifying resin having a Ring & Ball softening point of at least about 80° C. and up to about 140° C., wherein the viscosity of the composition is equal to or less than about 80,000 cP at 180° C. and the first polymer component, the second polymer component, and the tackifying resin are present in amounts effective to provide a hot melt adhesive composition which: (1) has a peel strength of at or above 100 grams-force at 1 gram per square meter both initially and after aging for 1 week and (2) a creep retention of at least 80% both initially and after aging for 1, 2, and 4 weeks. Embodiments of the invention function in a wide range of adhesive applications, including as elastic component adhesives and construction adhesives, and include specific adhesive formulations which are dually functional.

According to another embodiment of the present invention, a hot melt adhesive composition comprises (a) about 2% to about 50% by weight of a first polymer component having a low melting point and selected from the group consisting of a polypropylene homopolymer and a copolymer of propylene and ethylene and mixtures thereof; (b) about 2% to about 50% by weight of a second polymer component comprising an amorphous polyolefin; and (c) about 30% to about 75% by weight of a tackifying resin having a Ring & Ball softening point of at least about 80° C. and up to about 140° C., wherein the viscosity (measured by ASTM D3236-88) of the composition is equal to or less than about 80,000 cP at 180° C.

According to an embodiment of the invention, a method for making a laminate comprises the steps of: (a) applying a hot melt adhesive composition in a molten state to a primary substrate, wherein the hot melt adhesive composition comprises (i) about 2% to about 50% by weight of a first polymer component having a low melting point and selected from the group consisting of a polypropylene homopolymer and a copolymer of propylene and ethylene and mixtures thereof; (ii) about 2% to about 50% by weight of a second polymer component comprising an amorphous polyolefin; and (iii) about 30% to about 75% by weight of a tackifying resin having a Ring & Ball softening point of at least about 80° C. and up to about 140° C., wherein the viscosity (measured by ASTM D3236-88) of the composition is equal to or less than about 80,000 cP at 180° C.; (b) mating a secondary substrate to the first substrate by contacting the secondary substrate with the adhesive composition; and (c) cooling the adhesive.

Additional embodiments of the invention include the laminate made by a method of applying a hot melt adhesive composition according to the invention in a molten state to a primary substrate and mating a secondary substrate to the first substrate by contacting the secondary substrate with the adhesive, then cooling the adhesive. The laminate may be used as an elastic leg cuff, a standing leg cuff, or an elastic side panel in a disposable article. The laminate may also be used as part of a core of a disposable article in embodiments in which the adhesive is used as a construction adhesive; in that event, the laminate typically comprises a nonwoven substrate, the construction adhesive, and a backing layer or film, such as polyethylene film. Embodiments of the invention also include a disposable article, such as a diaper, comprising the adhesive of the present invention and at least one substrate.

A further embodiment of the invention is directed to a method for using a dually functional adhesive, comprising the steps of: (1) melting a single batch of an adhesive to form a molten adhesive; (2) dividing the molten adhesive into a first portion and a second portion; (3) directing the first portion to a first region of a plant and applying the adhesive at the first region to at least one of a first substrate or an elastic component to provide a first adhesive-bearing surface; (4) attaching the other of the first substrate or the elastic component to the first adhesive-bearing surface; (5) directing the second portion to a second region of the plant and applying the adhesive at the second region to at least one of a second substrate or a nonwoven layer to provide a second adhesive-bearing surface; and (6) attaching the other of the second substrate or the nonwoven layer to the second adhesive-bearing surface, wherein the adhesive is effective to provide: (1) a peel strength of at or above 100 grams-force at 1 gram per square meter both initially and after aging for 1 week and (2) a creep retention of at least 80% both initially and after aging for 1, 2, and 4 weeks. This embodiment permits the use of a single melt tank for an adhesive which can serve two end uses, optionally with the addition of plasticizer prior to application at one of such uses.

It is to be understood that both the foregoing general description and the following detailed description are exemplary, but are not restrictive, of the invention.

### BRIEF DESCRIPTION OF THE DRAWING

FIG. 1 shows peel performance, both initially and after various aging environments, of three exemplary formulations of the present invention, with each section of the graph showing, from left to right, the peel strength values for initial, one-week, two-week, and four-week aged values respectively.

FIG. 2 shows peel performance, both initially and after various aging environments, of an exemplary formulation of the present invention compared with another formulation.

### DETAILED DESCRIPTION OF THE INVENTION

According to an embodiment of the present invention, a hot melt adhesive composition comprises (a) about 2% to about 50% by weight of a first polymer component having a low melting point and selected from the group consisting of a polypropylene homopolymer and a copolymer of propylene and ethylene and mixtures thereof; (b) about 2% to about 50% by weight of a second polymer component comprising an amorphous polyolefin; and (c) about 30% to about 75% by weight of a tackifying resin having a Ring & Ball softening point of at least about 80° C. and up to about 140° C., wherein the viscosity (measured by ASTM D3236-88) of the composition is equal to or less than about 80,000 cP at 180° C.

Embodiments of the present invention are an adhesive based on mixtures of low melting point polypropylene polymers and amorphous alpha polyolefins along with a tackifying resin in an amount of at least about 30% by weight. (All percentages herein are by weight based on the total weight of the adhesive unless specifically noted otherwise.) Adhesives according to embodiments of the present invention exhibit excellent initial bonding with a variety of substrates, especially those which are elastomeric in nature, and provide bonds which are maintained upon long-term thermal aging making them useful for hygiene, construction, and packaging applications. Adhesives according to other embodiments of the invention provide good shear strength but additionally strong peel strength, particularly at low add-on levels, such as 1 or 2 grams per square meter. It has been found that certain adhesives of the present invention showing good creep resistance and peel strength are dually functional.

Generally, the hot melt adhesive composition of the invention comprises about 2% to about 50% by weight of a first polymer component having a low melting point and is selected from the group consisting of a polypropylene homopolymer and a copolymer of propylene and ethylene and mixtures thereof. As used herein, a “low melting point” of the first polymer component means that it has a melting point of less than 130° C., when measured using Differential Scanning calorimetry (DSC) according to ASTM E-794-01 except with one modification to the test in that a scanning temperature of 20° C. per minute instead of 10° C. per minute was used (the “DSC melting point”). Preferably, the DSC melting point of the first polymer is less than 95° C., more preferably less than 92° C., and most preferably less than 90° C. More preferably, the DSC melting point of the first polymer is at least 60° C., and more preferably at least 65° C. (When upper and lower limits of a range are separately provided herein to describe any feature or characteristic of the adhesives or constituents of the adhesives of the invention, aspects of the invention include ranges extending from any listed lower limit to any listed upper limit.)

According to an embodiment of the invention, the first polymer component has a low modulus, meaning that it can stretch to a relatively high extent before it breaks. One way to identify a polymer component which has a “low modulus” is to assess its elongation at break. In an embodiment of the invention, the first polymer component has an elongation at break of at least 20% according to ASTM D638 (defined herein as a “low modulus value”). Preferably, the first polymer component has a low modulus value of at least 100%, more preferably at least 150%, and most preferably at least 200%. Another way to measure a polymer component's modulus is to determine its elongation at break according to JIS-K 7113-2. In embodiments of the invention, the first polymer component has an elongation at break value according to JIS-K 7113-2 of at least 400%, more preferably at least 500%, and most preferably at least 550%.

The type and level of low melting point polypropylene-based polymers in the inventive formulations have been selected to provide the proper balance of flow needed for various application methods with the bond strength and ductility required to bind elastic components to a variety of substrates. Low melting point propylene-based polymers suitable for this application include propylene homopolymers that generally possess meso diad concentrations less than 90 mol % and DSC melting points below 130° C., preferably points below 100° C.

A new type of polyolefin has been developed by Idemitsu Petrochemical, Ltd. They have been described as their L-MODU grades, which is short for low molecular weight and low modulus polyolefin. Although they are entirely polypropylene based, they have properties not normally associated with polypropylene. Conventional polypropylene homopolymers tend to be very high in crystallinity and melting point. This is true whether or not they were prepared using Zeigler-Natta or metallocene catalysts technology. The new L-MODU grades are made using a unique metallocene catalyst which controls the stereoregularity of the polymer. This results in a new type of polymer which gives properties that were not attainable before. For example, the melting points of these new polymers are much lower than any other metallocene catalyzed polypropylene homopolymer. Typical polypropylene homopolymers have DSC melting points of about 130° C. to 170° C. The L-MODU polymers have Ring and Ball Softening points of under 130° C. when measured according to ASTM E-28-99. In an embodiment of the invention, the first polymer component is a polypropylene homopolymer and has a DSC melting point of less than 100° C.

The process to make these polymers is described in detail in U.S. Pat. No. 6,797,774 (assigned to Idemitsu Petrochemical Co., Ltd. Of Tokyo, JP). Because they have such low melting points and long recrystallization times, special considerations need to be taken into account to process them using underwater pelletizing equipment. This is described in U.S. Pat. No. 7,776,242 assigned to Idemitsu Kosan Co., Ltd. of Tokyo, JP. The disclosures found in U.S. Pat. No. 6,797,774 and U.S. Pat. No. 7,776,242 are both specifically incorporated into the present patent application by reference thereto. Certain characteristics of the Idemitsu L-MODU polypropylene homopolymers are listed below in Table 1.

TABLE 1

| | L-MODU | L-MODU | L-MODU |
|---|---|---|---|
| Properties | S400 | S600 | S901 |
| Density (kg/m³) | 870 | 870 | 870 |
| DSC Softening point (° C.)# | 78 | 78 | 79 |
| Molecular weight (weight average) | 45,000 | 70,000 | 120,000 |
| Molecular weight distribution | 2 | 2 | 2 |
| Tensile modulus (MPa) | 60 | 60 | 60 |
| Elongation at break (%) | 600 | 800 | 900 |
| Brookfield Melt viscosity at 190° | 9,000 | 52,000 | * |
| C. (cP) | | | |

#The DSC Softening points were run by Bostik's Analytical Laboratory. The other values were reported by Idemitsu on their web site.
* MFR = 50 g/10 min for 2.16 kg of L-MODU S901 at 230° C.

Even though the L-MODU polymers are polypropylene homopolymers, they are very different from traditional polypropylene polymers, as mentioned previously. Besides having much lower melting points when measured by DSC, their melt enthalpy values are also much lower than traditional polypropylene grades. When analyzed according to ASTM E793-01 “Standard Test Method for Enthalpies of Fusion and Crystallization by Differential Scanning calorimetry” except with the one modification that a scanning temperature of 20° C. per minute was used instead of 10° C. per minute, the following results shown in Table 2 were obtained.

TABLE 2

| | Glass Transition | | |
|---|---|---|---|
| L-MODU grade | Temperature (Tg) | Melt Peak | Melt Enthalpy |
| S-400 | −9.7° C. | 77.6° C. | 4.9 Joules/gram |
| S-600 | −7.8° C. | 77.1° C. | 22.6 Joules/gram |
| S-901 | −8.0° C. | 76.9° C. | 22.6 Joules/gram |

Both the melting points and melt enthalpies values are very low compared to most traditional polypropylene based homopolymers. Typical polypropylene homopolymers have melting points of from about 130° C. to 171° C. and melt enthalpy values of about 80 J/g or higher. The L-MODU polymers have a unique combination of melting point and melt enthalpy. However, we have found that to make a suitable hot melt adhesive using these materials as a base polymer requires the use of an additional polymer component as a second polymer component.

Other polymers may be used as the low melting point first polymer component and include: random poly-alpha-olefin copolymers and terpolymers derived of propylene with ethylene, butene, hexene, octene and combinations thereof. Some particularly preferred polyolefin polymers are copolymers of propylene with at least one other olefin monomer, such as ethylene-propylene copolymers and ethylene-octene copolymers. A preferred random copolymer includes propylene/ethylene elastomers, which can be obtained from ExxonMobil Chemical under the trade name designation Vistamaxx®. Suitable commercial grades range from about 5% to about 20% by weight ethylene, a melt flow rate of from about 1 to about 50 g/10 min, and a density of from about 0.84 to 0.88 grams/mL. One particularly preferred grade is Vistamaxx® 6202, which is a poly(propylene-co-ethylene) elastomer with about 85% propylene and 15% ethylene and has a melt mass-flow rate (230° C./2.16 kg) of 20 g/10 minutes and a density of 0.863 g/cc. A second preferred grade is Vistamaxx® 6502, which is a poly(propylene-co-ethylene) elastomer with about 87% propylene and 13% ethylene and has a melt mass-flow rate (230° C./2.16 kg) of 45 g/10 minutes and a density of 0.865 g/cc.

The first polymer component is generally present in the adhesive compositions for any use in amounts of about 2% to about 50%, preferably about 5% to about 45%, and most preferably about 7.5% to about 40%, by weight. Mixtures of polypropylenes at these levels are also suitable. From about 5% to about 30% by weight of one or more additional polymers may be blended together with the first polymer if desired. The weight average weight molecular weight of the first polymer component according to embodiments of the present invention may be in the range of from about 2,000 gram/mol to about 150,000 g/mol, preferably from about 20,000 g/mol to about 150,000 g/mol. The values above are ranges for the use of the adhesive generally. In preferred embodiments in which the adhesive is used as an elastic component adhesive, the first polymer component may be present in the adhesive composition in amounts of about 5% to about 35%, more preferably about 10% to about 30% by weight, and most preferably from about 15% to about 25% by weight. In preferred embodiments in which the adhesive is used as a construction adhesive, the first polymer component may be present in the adhesive composition in amounts of about 15% to about 38%, more preferably about 18% to about 33% by weight, and most preferably from about 20% to about 32% by weight. In preferred embodiments in which the adhesive is dually functional (i.e., can be used as either a construction adhesive or as an elastic component adhesive), the first polymer component may be present in the adhesive composition in amounts of about 5% to about 30%, more preferably about 8% to about 25% by weight, and most preferably from about 10% to about 17% by weight.

The hot melt adhesive composition of the present invention also includes a second polymer component comprising an amorphous polyolefin present at about 2% to about 50% by weight for any use. The presence of this second polymer component, such as an amorphous poly-alpha-olefin (APAO), is believed to provide cohesive strength as well as modify the ultimate physical properties of the adhesive. In particular, the combination of APAO polymers with the low-modulus, low melting point first polymer component described above in combination with a judiciously selected amount of tackifying agent has been shown to afford adhesives with the cohesive strength required to maintain strong bonds to stressed elastic components as full properties of the adhesive develop over time. Unlike other materials such as higher crystallinity polyolefins or polyolefin waxes that can be envisioned to provide similar set up benefits, APAO materials are believed to offer enhanced compatibility with the other key components of the inventive formulation to enhance long-term phase stability of the adhesive.

The second polymer component of the blend useful in the present invention includes several different categories of low molecular weight, low melt viscosity, and amorphous propylene-containing polymers. The term “amorphous” is defined herein as having a degree of crystallinity less than 30%, as determined by differential scanning calorimetry (DSC) against a highly crystalline polypropylene standard. These polymers can be either homopolymers of propylene or copolymers of propylene with one or more alpha-olefin (1-alkene) comonomer, such as, for example, ethylene, 1-butene, 1-hexene, and 1-octene. Poly(1-butene-co-propylene) polymers referred to as “butene rich” APAO polymers are also suitable for the present invention. The polymers advantageously display Ring & Ball softening points between about 80° C. and 170° C. according to ASTM E28 and a glass transition temperature from about −5° C. to −40° C. according to ASTM D3417.

In one embodiment, the amorphous polymers are poly-alpha olefin polymers that have a melt viscosity range greater than about 500 cP to about 120,000 cP and more preferably 500 cP to 8,000 cP at 190° C. (determined in accordance with ASTM D3236).

Preferably the second polymer component comprises a poly-α-olefin, preferably an amorphous poly-α-olefin. Preferred second polymer components comprise “propylene rich” poly(l-propylene-co-1-butene) copolymers and/or amorphous polypropylene co- and terpolymers of ethylene and/or 1-butene. Exemplary amorphous poly-alpha olefin copolymers include the REXtac® 2830 from REXtac LLC and Vestoplast® EP NC 702 from series from Evonik Industries.

The second polymer component is generally present in the adhesive compositions for any use in the amounts of 2% to about 50%, preferably about 5% to about 40%, and most preferably about 5% to about 30%, by weight. The values above are ranges for use of the adhesive generally. In preferred embodiments in which the adhesive is used as an elastic component adhesive, the second polymer component may be present in the adhesive composition in amounts of about 10% to about 45%, more preferably about 20% to about 40% by weight, and most preferably from about 25% to about 35% by weight. In preferred embodiments in which the adhesive is used as a construction adhesive, the second polymer component may be present in the adhesive composition in amounts of about 10% to about 40%, more preferably about 15% to about 35% by weight, and most preferably from about 20% to about 28% by weight. In preferred embodiments in which the adhesive is dually functional (i.e., can be used as either a construction adhesive or as an elastic component adhesive), the second polymer component may be present in the adhesive composition in amounts of about 15% to about 40%, more preferably about 18% to about 32% by weight, and most preferably from about 20% to about 26% by weight.

A tackifying resin, as defined in the present description can be a molecule or a macro-molecule, generally a chemical compound or a fairly low molecular weight polymer, compared to common polymers, from a natural source or from a chemical process or combination thereof that in general enhances the adhesion of a final hot melt adhesive composition. Representative resins include the C5/C9 hydrocarbon resins, synthetic polyterpenes, rosin, rosin esters, natural terpenes, and the like. More particularly, the useful tackifying resins include any compatible resins or mixtures thereof such as (1) natural and modified rosins including gum rosin, wood rosin, tall oil rosin, distilled rosin, hydrogenated rosin, dimerized rosin, and polymerized rosin; (2) glycerol and pentaerythritol esters of natural and modified rosins, including the glycerol ester of pale, wood rosin, the glycerol ester of hydrogenated rosin, the glycerol ester of polymerized rosin, the pentaerythritol ester of hydrogenated rosin, and the phenolic-modified pentaerythritol ester of rosin; (3) copolymers and terpolymers of natural terpenes, such as styrene/terpene and alpha methyl styrene/terpene; (4) polyterpene resins generally resulting from the polymerization of terepene hydrocarbons, such as the bicyclic monoterpene known as pinene, in the presence of Friedel-Crafts catalysts at moderately low temperatures; also included are the hydrogenated polyterpene resins; (5) phenolic modified terpene resins and hydrogenated derivatives thereof such, for example, as the resin product resulting from the condensation, in an acidic medium, of a bicyclic terpene and a phenol; (6) aliphatic petroleum hydrocarbon resins resulting from the polymerization of monomers consisting primarily of olefins and diolefins; also included are the hydrogenated aliphatic petroleum hydrocarbon resins; and (7) cyclic petroleum hydrocarbon resins and the hydrogenated derivatives thereof. Mixtures of two or more of the above described tackifying resins may be required for some formulations. Also included are the cyclic or acylic C5 resins and aromatic modified acyclic or cyclic resins.

In an embodiment of the invention, the tackifier is selected from the group consisting of aliphatic and cycloaliphatic hydrocarbon resins and their hydrogenated derivatives, hydrogenated aromatic hydrocarbon resins, aromatically modified aliphatic or cycloaliphatic resins and their hydrogenated derivatives, polyterpene and styrenated polyterpene resins and mixtures thereof. In another embodiment of the invention, the tackifier is selected from the group consisting of a C-5 aliphatic hydrocarbon resin, a hydrogenated C-5 resin, a hydrogenated C-9 resin, a hydrogenated DCPD resin and an aromatic-modified DCPD resin.

In an embodiment of the invention, the tackifying resin has a Ring and Ball softening point (measured by ASTM E28) of at least about 40° C., most preferably between about 80° C. and 140° C. A preferred tackifier possesses Ring and Ball softening point (RBSP) between about 85° C. to 135° C. and can be obtained from ExxonMobil Chemical under the tradename of Escorez 5400, 5600, and 5615. One preferred tackifying agent is Sukorez SU-210 which a hydrogenated C5/cyclic hydrocarbon resin with a RBSP of between 107-114° C. available from Kolon. Other preferred tackifying resins are available from Eastman Chemical Company and include, but not limited to: partially hydrogenated aliphatic hydrocarbon resins such as Eastotac® H100L and Eastotac® H100R, as well as non-hydrogenated aliphatic C5 resins and aromatic modified C5 resins with low aromaticity such as Piccotac® 1095 and Piccotac® 9095, respectively.

An embodiment of the present invention provides a hot melt adhesive composition for any use comprising a tackifying resin in an amount of from about 30 to about 75% by weight. For preferred performance for use generally and as an elastic component adhesive, tackifiers may be present in the adhesive compositions in amount of about 30 to 75% by weight of the composition, preferably about 32% to 73%, more preferably about 35 to 70% by weight, and most preferably about 45 to 65% by weight. In preferred embodiments for use of the adhesive as a construction adhesive, tackifiers may be present in the adhesive composition in amount of about 30 to 60% by weight of the composition, preferably about 32% to 55%, more preferably about 34 to 50% by weight, and most preferably about 35 to 45% by weight. In preferred embodiments in which the adhesive is dually functional (i.e., can be used as either a construction adhesive or as an elastic component adhesive), the tackifier may be present in the adhesive composition in amounts of about 30% to about 70%, more preferably 32%, 34%, 36%, 38%, or 40% to about 60% by weight, and most preferably from about 45% to about 55% by weight. Blends of two or more tackifying resins may also be used. For example, a blend of a first tackifying resin and a second tackifying resin that is different than the first tackifying resin may also be employed. From about 5% to about 70% by weight of one or more additional tackifying resins may be blended together with the first tackifying resin if desired.

Plasticizers may also be used in the present invention to control the behavior of the adhesive during application and end-use. The plasticizer component useful in the present invention may be selected from any of the mineral based oils, petroleum based oils, liquid resins, liquid elastomers, polybutene, polyisobutylene, phthalate and benzoate plasticizers, and epoxidized soya oil. Preferably, the plasticizer is selected from the group consisting of mineral oil and liquid polybutene, and even more preferably mineral oil with less than 30% aromatic carbon atoms. A plasticizer is broadly defined as a typically organic composition that can be added to the thermoplastic rubbers and other resins to improve extrudability, flexibility, workability and stretchability in the finished adhesive. Any material which flows at ambient or application temperatures and is compatible in the compositions of the present invention may be useful. Preferably, the plasticizer has low volatility at temperatures of greater than about 40° C. The most commonly used plasticizers are oils which are primarily hydrocarbon oils, low in aromatic content and are paraffinic or naphthenic in character. The oils are preferably low in volatility, transparent and have as little color and negligible odor. This invention also may include olefin oligomers, low molecular weight polymers, synthetic hydrocarbon oils, vegetable oils and their derivatives and similar plasticizing oils. Solid plasticizers may also be useful to the present invention. Examples of such plasticizers include 1,4-cyclohexane dimethanol dibenzoate, glyceryl tribenzoate, pentaerythritol tetrabenzoate, and dicylcohexylphthalate. Preference is given to the petroleum based oils with suitable naphthenic minerals oils useful in this invention of the types herein described above are commercially available from Nynas, under the trade name Nyplast®. Suitable liquid plasticizers include polybutene such as Indopol series materials supplied by Ineos. As required, blends of plasticizers can also be employed to adjust end use performance and final properties.

If used in embodiments in which the adhesive is suitable for use as an elastic component adhesive, the plasticizer may be used in an amount of about 0.1% to about 20%, more preferably about 0.5% to about 15%, by weight of the adhesive. In some embodiments, no plasticizer is used. For embodiments in which the adhesive is suitable for use as a construction adhesive, the plasticizer may be used in an amount of about 1% to about 25%, more preferably about 5% to about 20%, and most preferably about 8% to about 17% by weight of the adhesive. In preferred embodiments in which the adhesive is dually functional (i.e., can be used as either a construction adhesive or as an elastic component adhesive), the plasticizer may be present in the adhesive composition in amounts of about 5% to about 20%, more preferably about 8% to 20% by weight, and most preferably from about 12% to 16%, 16.5%, 17%, 17.5%, or 18% by weight. Blends of two or more plasticizers may also be used. For example, a blend of a first plasticizer and a second plasticizer that is different than the first plasticizer may also be employed. From about 1% to about 19% by weight of one or more additional plasticizers may be blended together with the first plasticizers if desired, to achieve the totals listed above.

The present invention may include a stabilizer or an antioxidant in an amount of from about 0% to about 5% by weight. Preferably from about 0.1% to 2% of a stabilizer or antioxidant is incorporated into the composition. The stabilizers which are useful in the hot melt adhesive compositions of the present invention are incorporated to help protect the polymers noted above, and thereby the total adhesive system, from the effects of thermal and oxidative degradation which normally occurs during the manufacture and application of the indicator as well as in the ordinary exposure of the final product to the ambient environment. Among the applicable stabilizers are hindered phenols and multifunction phenols, such as sulfur and phosphorous-containing phenols. Antioxidants, such as hindered amine phenols, may be characterized as phenolic compounds that also contain bulky radicals in close proximity to the phenolic hydroxyl group thereof and are preferred. In particular, tertiary butyl groups generally are substituted onto the benzene ring in at least one of the ortho positions relative to the phenolic hydroxyl group. The presence of these sterically bulky substituted radicals in the vicinity of the hydroxyl group serves to retard its stretching frequency and correspondingly, its reactivity; this steric hindrance thus providing the phenolic compound with its stabilizing properties. Representative hindered phenols include:
1,3,5-trimethyl-2,4,6-tris(3-5-di-tert-butyl-4-hydroxybenzyl) benzene;
pentaerythritol tetrakis-3(3,5-di-tert-butyl-4-hydroxyphenyl)propionate;
n-octadecyl-3(3,5-di-tert-butyl-4-hydroxyphenyl) propionate;
4,4′-methylenebis(4-methyl-6-tertbutylphenol);
2,6-di-tert-butylphenol;
6-(4-hydroxyphenoxy)-2,4-bis(n-octylthio1,3,5-triazine;
2,3,6-tris(4-hydroxy-3,5-di-tert-butyl-phenoxy,3,5-triazine
di-n-octadecyl-3,5-di-tert-butyl-4-hydroxybenzylphosphonate;
2-(n-octylthio)ethyl-3,5-di-tert-butyl-4-hydroxybenzoate; and
sorbitol hexa-3 (3,5-di-tert-butyl-4-hydroxy-phenyl)propionate.

Polyolefin nucleating agents may also be also present in the invention. Nucleating agents suitable for this invention are generally of the sub class of nucleating agents known as clarifying agents that are commonly employed in polyolefins additive packages to promote rapid crystallization. Suitable materials include dibenzylidene sorbitol derivatives such as Millad 3988 and Millad NX8000 supplied by Milliken as well as Irgaclear D produced by BASF. Other suitable agents include aromatic amide systems such as NJ Star NU-100 provided by New Japan Chemical Company.

If included, the nucleating agent is generally present in the adhesive compositions in amounts of about 0.05 to 5% by weight of the composition, preferably about 0.1 to 2.5% by weight are utilized, and most preferably about 0.2 to 1.0% by weight. Blends of two or more nucleating agent may also be used. For example, a blend of a nucleating agent and a second nucleating agent that is different than the first nucleating agent may also be employed. From about 0.05% to about 5% by weight of one or more additional nucleating agent may be blended together with the first nucleating agent if desired. The nucleating agent may be used directly as a powder, as a slurry in a portion of suitable plasticizing agent, or as a component in a masterbatch of suitable polymer masterbatch such as Milliken NX-10. Nucleation packages such as those described in US 2015/0299526 can also be included to tailor the set up rate and bonding properties of the hot-melt adhesive.

In addition to the polymer components in the present adhesive composition, about 1% to about 15% by weight of an additional auxiliary polymer selected from the group consisting of ethylene-vinyl acetate (EVA), polyethylene (PE), low-density polyethylene (LDPE), linear low-density polyethylene(LLDPE), polybutylene (PB), and a styrenic block copolymer and mixtures thereof, may also be used. The auxiliary polymer may be a styrene block copolymer selected from the group consisting of styrene-isoprene-styrene (SIS), styrene-isoprene (SI), styrene-butadiene-styrene (SBS), styrene-butadiene (SB), styrene-isoprene-butadiene-styrene (SIBS), styrene-ethylene-butadiene (SEB), styrene-ethylene-butadiene-styrene (SEBS), styrene-ethylene-propylene (SEP), styrene-ethylene-propylene-styrene (SEPS), styrene-ethylene-ethylene-propylene-styrene (SEEPS) and blends of each thereof. The auxiliary polymer is a polymer that is different from the first polymer component, the second polymer component, and the tackifying resin, and functions to provide a desired physical property, depending on the end use of the adhesive composition.

It should be understood that other optional additives may be incorporated into the adhesive composition of the present invention in order to modify particular physical properties. These may include, for example, such materials as ultraviolet light (UV) absorbers, waxes, surfactants, inert colorants, titanium dioxide, fluorescing agents and fillers. Typical fillers include talc, calcium carbonate, clay silica, mica, wollastonite, feldspar, aluminum silicate, alumina, hydrated alumina, glass microspheres, ceramic microspheres, thermoplastic microspheres, baryte and wood flour and may be included in an amount up to 60% by weight, and preferably between 1 and 50% by weight.

Of these optional additives, waxes may be included in the amount up to 20% by weight, preferably between 0.1% and 20% by weight. In an embodiment of the invention, the wax is selected from the group consisting of petroleum waxes, low molecular weight polyethylene and polypropylene, synthetic waxes and polyolefin waxes and mixtures thereof. In preferred embodiments, the wax is a low molecular weight polyethylene having a number average molecular weight of about 400 to about 6,000 g/mol.

The viscosity of the adhesive material according to the present invention should be generally at a viscosity at the application temperature appropriate to be processed and applied to the substrate that it is being applied to. An adhesive with relatively low viscosity is needed to be processed through standard hot melt adhesive equipment and to achieve the desired pattern and consequently suitable bonding performance at the application temperature. In general, the viscosity is equal to or lower than 80,000 centipoise (cP), and most preferably lower than 40,000 cP measured at 180° C. (356° F.) according to ASTM D 4287-00) (except that reading are taken at five minutes instead of after 15 seconds or less). All viscosities identified herein are measured according to this modified ASTM standard. Preferably, the viscosity of the composition is equal to or less than about 80,000 cP at 180° C. (356° F.), and most preferably equal to or less than 40,000 cP at 180° C. Preferably, the viscosity of the composition is at least 1,000 cP, more preferably 2,500 cP, still more preferably at least 5,000 cP, and most preferably at least 15,000 cP, all at 180° C. The above values are suitable for the adhesive generally and when used as an elastic component adhesive. In embodiments in which the adhesive is suitable for use as a construction adhesive, the viscosity of the composition is at least 500 cP, more preferably between 1,000 cP and 8,000 cP, still more preferably between about 2,000 cP and 6,000 cP, and most preferably between about 3,000 cP and 4,000 cP, all at 148.9° C., according to ASTM D 4287-00 (except that readings are taken at five minutes instead of after 15 seconds or less).

In embodiments of the invention, the hot melt adhesive composition consists essentially of, or consists of, first polymer component, the second polymer component, the tackifying resin, and, optionally, the plasticizer. In some embodiments, the hot melt adhesive composition does not include a wax.

The hot melt adhesive composition of the present invention may be formulated using any of the techniques known in the art. A representative example of the mixing procedure involves placing all the components in a jacketed mixing vessel equipped with a rotor, and thereafter raising the temperature of the mixture to a range from 120 to 230° C. to melt the contents. It should be understood that the precise temperature to be used in this step would depend on the melting points of the particular ingredients. The constituents are individually or in certain combinations introduced to the vessel under agitation and the mixing is allowed to continue until a consistent and uniform mixture is formed.

In an embodiment of the invention, the adhesive is made using a traditional overhead mixer at 176.7° C. First, the plasticizer, tackifier, and any antioxidant(s) are heated to desired temperature and stirring is started for homogeneity. The order of polymer addition does not appear to impact the final result, although in some embodiments the first polymer component is added first. After all polymer is dissolved and the mix appears homogenous, the viscosity can be tested. The contents of the vessel may be protected with inert gas, such as nitrogen, during the entire mixing process. Other conventional methods may be used to make the hot melt adhesive of the present invention. For example, methods employing static mixing, single screw extrusion, twin screw extrusion, and kneading, may be used. The hot melt adhesive is then cooled to room temperature and formed into chubs with a protective skin formed thereon or into pellets for shipment and use.

The resulting hot melt adhesive may then be applied to substrates using a variety of coating techniques. Examples include hot melt slot die coating, hot melt wheel coating, hot melt roller coating, melt-blown coating as well as slot, spiral spray, and wrapping spray methods such as those used to affix elastic strands. Spray techniques are numerous and can be done with or without assistance of compressed air that would shape the adhesive spray pattern. The hot melt adhesive material is generally pumped molten through hoses to the final coating spot on the substrates. Any application temperature above the softening point of the adhesive formulation is suitable.

The adhesive composition of the present invention may be used in a number of applications such as, for example, in disposable nonwoven hygienic articles, paper converting, flexible packaging, wood working, carton and case sealing, labeling and other assembly applications. Particularly preferred applications include diaper and adult incontinent brief elastic attachment, disposable diaper and feminine sanitary napkin construction, diaper and napkin core stabilization, diaper backsheet lamination, industrial filter material conversion, surgical gown and surgical drape assembly.

The adhesive of the present invention can be used with any application where various substrate materials are involved. Examples include nonwoven materials, polymeric films, and, in general, elastomeric components put in items such as diapers, in the form of strands, films, elastic cuffs, webs, scrims, nonwovens or any other continuous or discrete form. Any substrate material and any substrate form could be used in any combination possible with the adhesive serving to bond a single substrate folded over on itself or two or more substrates together. The substrates can be of multiple forms, for example fiber, film, thread, strip, ribbon, tape, coating, foil, sheet, and band. The substrate can be of any known composition for example polyolefin, polyacrylic, polyester, polyvinyl chloride, polystyrene, cellulosic like wood, cardboard or paper. The bulk substrate's mechanical behavior can be rigid, plastic, or elastomeric. For example, the adhesive can be employed to apply elastic fibers to supple materials such as nonwovens or plastic films. Among elastomeric materials are various examples like natural or synthetic rubber, polyurethane based copolymers, polyether or polyester urethanes, block copolymers of styrene or of amides, or olefinic copolymers. The above lists are not limitative or all-inclusive, but are only provided as common examples.

The adhesive of the present invention can also be used in any application where composites and disposable products are made by bonding substrates together while obtaining adequate cohesion from the adhesive bond to withstand mechanical stress at low, ambient, and/or elevated temperatures, in particular under shear conditions. Diapers, adult incontinence products, sanitary napkins and other absorbent disposable products are potential applications for the adhesive composition of the invention, as well as bed pads, absorbing pads, surgical drapes and other related medical or surgical devices. The adhesives described here are especially useful in elastic attachment applications in disposable diapers and other hygiene products.

In an embodiment of the invention, a method of making a laminate comprises the steps of: (1) applying the hot melt adhesive composition of the invention in a molten state to a first substrate; and (2) mating a secondary substrate to the first substrate by contacting the secondary substrate with the adhesive composition and then allowing the adhesive to cool. In embodiments in which the adhesive is suitable for use as an elastic component adhesive, the first substrate may be an elastic portion of a diaper, such as an elastic strand used as part of a leg cuff of a diaper. It is preferably in a stretched state when the adhesive is applied. Such elastic strands (or bands) and their application as part of a leg cuff of a diaper are shown in U.S. Pat. No. 5,190,606, incorporated herein by reference. The secondary substrate may comprise a nonwoven material or a film, such as a SMS nonwoven fabric polyethylene film, and the method may include folding the secondary substrate around the elastic strand. In this way, only the secondary substrate may serve as the substrate which encapsulates the strand or strands of the leg cuff. In an alternative embodiment, a tertiary substrate is used, and the secondary and tertiary substrates may be mated to the elastic strand on opposite sides of the elastic strand. In such an embodiment, the secondary substrate may be a polyethylene film and the tertiary substrate may be a film of nonwoven material, or verse visa. Furthermore, a composite diaper backsheet comprising a thermoplastic film joined to a nonwoven fabric can also be used as the secondary and tertiary substrates mentioned above.

In embodiments in which the adhesive is suitable for use as a construction adhesive, the first substrate may be a polyolefin film and the second substrate may be a nonwoven material or film.

In alternative embodiments of the invention, the adhesive is applied to the first substrate using a direct contact method of hot melt application, such as a slot or V-slot applicator head. Alternatively, the adhesive may be applied to the first substrate using a non-contact method of hot melt, such as a spray applicator. The first substrate, to which the adhesive in a molten state is applied, may be an elastic strand or a nonwoven fabric. In embodiments in which the first substrate is an elastic strand, the secondary substrate may be a nonwoven fabric wrapped around the elastic strand, or the secondary substrate could alternatively be elastic between two layers of nonwoven. In such embodiments, the laminate made by the method may be used as an elastic leg cuff, standing leg cuff, or elastic side panel in a disposable article, such as a diaper.

According to embodiments of the invention, a hot melt adhesive composition suitable for use as both an elastic component adhesive and a construction adhesive comprises (a) about 2% to about 50% by weight of a first polymer component having a low melting point and selected from the group consisting of a polypropylene homopolymer and a copolymer of propylene and ethylene and mixtures thereof; (b) about 2% to about 50% by weight of a second polymer component comprising an amorphous polyolefin; and (c) about 30% to about 75% by weight of a tackifying resin having a Ring & Ball softening point of at least about 80° C. and up to about 140° C., wherein the viscosity (measured by ASTM D3236-88) of the composition is equal to or less than about 80,000 cP at 180° C., and wherein the weight ratio of the first polymer component to the second polymer component varies from about 1:3 to about 5:4, preferably from about 1:2 to about 1:1, more preferably from about 2:3 to about 99:100 and most preferably from about 3:4 to about 24:25. For embodiments in which the adhesive is dually functional, the weight ratio of the first polymer component to the second polymer component varies from about 1:5 to about 1:1, preferably from about 3:10 to about 9:10, more preferably from about 2:5 to about 4:5 and most preferably from about 1:2 to about 7:10. More preferably, the weight ratio of total polymer to tackifying resin varies from about 3:7 to about 7:3, preferably from about 2:3 to about 7:4, more preferably from about 5:6 to about 7:5 and most preferably from about 1:1 to about 5:4. For embodiments in which the adhesive is dually functional, the weight ratio of total polymer:tackifying resin may vary from about 1:3 to about 3:2, more preferably from about 2:5 to about 9:8, and most preferably from about 3:5 to about 5:6. In some embodiments, the first polymer component is a propylene-co-ethylene polymer and the second polymer component is a APAO, preferably a butene-rich APAO. In an embodiment in which the adhesive is dually functional, the first polymer component is present in an amount of from about 5% to about 30% by weight, the second polymer component is present in an amount of from about 15% to about 40%, the tackifying resin is present in an amount of from about 30% to about 70% by weight, and the plasticizer is present in the adhesive composition in amounts of from about 5% to about 20% by weight.

According to embodiments of the invention, a hot melt adhesive composition suitable for use as both an elastic component adhesive and a construction adhesive comprises (a) a first polymer component having a low melting point and selected from the group consisting of a polypropylene homopolymer and a copolymer of propylene and ethylene and mixtures thereof; (b) a second polymer component comprising an amorphous polyolefin; and (c) a tackifying resin having a Ring & Ball softening point of at least about 80° C. and up to about 140° C., wherein the viscosity (measured by ASTM D3236-88) of the composition is equal to or less than about 80,000 cP at 180° C., and wherein the first polymer component, the second polymer component, and the tackifying resin are present in amounts effective to provide a hot melt adhesive composition which: (1) has a peel strength of at or above 100 grams-force at 1 gram per square meter both initially and after aging for 1 week and (2) a creep retention of at least 80% both initially and after aging for 1, 2, and 4 weeks. In this embodiment, peel strength and creep retention are determined as set forth in the examples herein. In particular, initial and aged creep retention is determined as set forth in Example 1 below (with an add-on level target of 35 mg adhesive/m strand). The peel strength is determined as set forth in the description of Examples 8-12 below, except having an add-on of one gram per square meter.

In still another embodiment of the invention, a method for using a dually functional adhesive comprises the steps of: (1) melting a single batch of an adhesive to form a molten adhesive; (2) dividing the molten adhesive into a first portion and a second portion; (3) directing the first portion to a first region of a plant and applying the adhesive at the first region to at least one of a first substrate or an elastic component to provide a first adhesive-bearing surface; (4) attaching the other of the first substrate or the elastic component to the first adhesive-bearing surface; (5) directing the second portion to a second region of the plant and applying the adhesive at the second region to at least one of a second substrate or a nonwoven layer to provide a second adhesive-bearing surface; and (6) attaching the other of the second substrate or the nonwoven layer to the second adhesive-bearing surface, wherein the adhesive is effective to provide: (1) a peel strength of at or above 100 grams-force at 1 gram per square meter both initially and after aging for 1 week and (2) a creep retention of at least 80% both initially and after aging for 1, 2, and 4 weeks. In this embodiment, peel strength and creep retention are determined as set forth in the examples herein. In particular, initial and aged creep retention is determined as set forth in Example 1 below (with an add-on level target of 35 mg adhesive/m strand). The peel strength is determined as set forth in the description of Examples 8-12 below, except having an add-on of one gram per square meter.

In this embodiment, the different “regions” of a plant include different areas at which different steps of applying an adhesive are carried out, such as different sections of a diaper fabrication line. For example, the first region of a plant may be the section of a line at which the adhesive is applied to an elastic component or the substrate to which the elastic component is attached (or both), at which the adhesive is used as an elastic component adhesive. Further, the second region of a plant may be the section of a line at which the adhesive is applied to a nonwoven layer or the substrate to which the nonwoven is attached (or both), at which the adhesive is used as a construction adhesive. In an embodiment of the invention, the same base adhesive may be used for each end use, but a plasticizer may be added to the base adhesive before applying the adhesive at the second region to the second substrate or the nonwoven layer. Thus, an embodiment of the invention comprises adding a plasticizer to the second portion of the adhesive before the adhesive is applied at the second region to the second substrate or the nonwoven layer.

### EXAMPLES

The following examples are illustrative but not limiting of the invention.

Viscosity was measured according to ASTM D 4287-00) (except that readings were taken at five minutes instead of after 15 seconds or less). Measurements were conducted at 162.8° C. unless otherwise noted. Approximately 0.13 g of sample was placed in the center of the plate and the cone (Spindle 09) was slowly lowered until sample was fully melted. The test was started after the temperature had stabilized at the target (approximately five minutes). The spindle speed was adjusted so the percent torque was between 45% and 90%. After the starting test was run for five minutes, the viscosity reading was recorded.

Ring & Ball softening points were determined with an automated Herzog unit according to the method set forth in ASTM E-28.

Raw Materials:

Escorez 5400 is a hydrogenated cycloaliphatic hydrocarbon resin with a 103° C. softening point. It is available from ExxonMobil Chemical.

Escorez 5615 is a cycloaliphatic hydrocarbon resin with a 118° C. softening point. It is available from ExxonMobil Chemical.

Nyflex 222B is an oil used as a plasticizer comprising 55% paraffinic and 44% naphthenic. It is commercially available from Nynas AB.

Sukorez SU-210 is a hydrogenated hydrocarbon tackifying resin produced by Kolon Chemicals.

Sukorez SU-100 is a hydrogenated dicyclopentadiene (DCDP) hydrocarbon resin used as a tackifying resin and is produced by Kolon Chemicals.

L-MODU 5400, L-MODU 5600, and L-MODU 5901 are low modulus, controlled tacticity polypropylenes available from Idemitsu.

Rextac 2330 is a propylene/ethylene copolymer with a Brookfield viscosity of 3,000 cP at 190° C. (374° F.) and a Ring & Ball Softening Point of 141° C. (286° F.). It is available from REXtac, LLC in Odessa, Tex.

Rextac 2830 is a butene-1 rich grade of APAO. It has a viscosity of 3,000 cP at 190° C. (374° F.) and a R&B Softening Point of 90° C. (200° F.). It is available from REXtac, LLC in Odessa, Tex.

Vestoplast 508 is a butene-1 rich grade of APAO with a viscosity of 8,000 cP at 190° C. and a R&B Softening Point of 84° C. (183° F.). It is available from Evonik Industries AG.

Vestoplast EP NC 702 is a propene rich APAO polymer with a viscosity of 2,800 cP at 190° C. and a R&B Softening Point of 105° C. (221° F.). It is available from Evonik Industries AG.

Vistamaxx 6202 is a propylene-co-ethylene polymer available from Exxon Mobil Corporation.

Irganox 1010 antioxidant is pentaerythritol tetrakis(3-(3,5-di-tert-butyl-4-hydroxyphenyl)propionate) available from BASF.

Irgafos 168 antioxidant is hydrolytically stable phosphite available from BASF.

Evernox 1010 is a phenolic antioxidant for thermal stabilization.

BAS 450 SD is a heterophasic impact polypropylene type material available from Borealis.

### Examples 1-7—Testing in Elastic Attachment Applications

The adhesives were coated at 325° F. in a continuous fashion to Invista 680 dtex elastic strands elongated to 300% their unstrained length. Except when described otherwise, coating was performed using a Nordson Allegro slot-type applicator operated within specifications described by the manufacturer and an add-on level target of 35 mg adhesive/m strand. For all tests, three elastic strands spaced 5 mm apart were laminated between a nonwoven substrate (FQN, 3.5″ width, basis weight 15 g/m²) and a breathable PE film (Clopay BR 134, 2.75″ width). The line speed was 900 ft/min and nip pressures of 40 psi were used to compress the elastic, nonwoven, and PE film. The final laminate structure was spooled on a take up roll in elongated fashion during each trial. Immediately following the relatively short—less than five minute—production runs, a portion of the final laminate was collected and allowed to stand in the relaxed (unspooled) state prior to testing.

Creep resistance was measured to gauge the ability of the inventive formulations to bond elastic strands. In this testing, a 500 mm length of the fabricated laminate article was stretched across a Plexiglas board until the nonwoven and PE film substrates were fully elongated, but not deformed beyond the stress yield of the materials. The ends of the laminate article were secured to the board to maintain. Next, a pen was used to mark across the elastics at 100 mm and 400 mm so that a 300 mm long segment of stretched laminate was designated as the test area. Each of the elastic strands were cut at the lines on the 100 mm and 400 mm marks and the board with strained laminated article was then placed in an oven at 37.8° C. After four hours, the structure was removed from the oven and the ends of the cut elastic strands were marked with a pen. The creep retention, expressed as a percentage, is a measured of the length of the cut strands after exposure to 37.8° C. for four hours divided by the original length of the section cut (300 mm). Aged creep resistance was calculated by conditioning a portion of the laminated structure to an elevated temperature (54.4° C.) for a designated period of time (1, 2, or 4 weeks) and then tested according to the initial creep resistance test.

To gauge off-line performance, green creep resistance was also recorded on samples taken immediately after fabrication. In these tests, the laminated structure was fully elongated as described above and a single line was marked in the middle of the specimen. The elastic strands were cut and after 30 minutes green creep was determined as the length (mm) the cut end of the elastic strand moved from the middle mark. A green creep of 5 mm or less is considered acceptable.

Synthetic Procedure:

All formulations were produced on a ca. 2.5 kg scale, using the following method. A 3.875 L stainless-steel vessel was charged with the tackifying, APAO, and antioxidant. A digitally-controlled heating mantel equipped with an internal thermocouple was used to gradually heat the formulation to the target temperature (170° C. to 190° C.). After the mixture appeared molten, the solution was mechanically stirred between 100 rpm to 200 rpm using a pitched-blade impeller. Next, the low melting point polyolefin was gradually added to the stirred mixture. The resultant clear to slightly hazy molten mixture was held at the target temperature an additional 60 minutes to 240 minutes until it appeared to be fully homogenized. After this time, the vessel was removed from the heating mantel and samples were collected for testing.

Tables 3 and 4 provide physical property data, as well as Green and Aged Creep Retention Performance for the inventive formulations (Examples 1, 2, 3, 4, 5, 6, and 7) as well as comparative examples based on prior art.

TABLE 3
Formulas, Properties, and Creep Performance
of Inventive and Comparison Examples

| Example | 1 | 2 | 3 | CE1 | CE2 |
|---|---|---|---|---|---|
| Escorez 5400, wt % | 59.5 | 49.5 | 39.5 | 29.5 | 19.5 |
| Sukorez SU-210, wt % | — | — | — | — | — |
| Rextac 2830, wt % | 20.0 | 30.0 | 40.0 | 50.0 | 60.0 |
| L-MODU S600, wt % | 20.0 | 20.0 | 20.0 | 20.0 | 20.0 |
| L-MODU S901, wt % | — | — | — | — | — |
| Vistamaxx 6202, wt % | — | — | — | — | — |
| Irganox 1010, wt % | 0.5 | 0.5 | 0.5 | 0.5 | 0.5 |
| RBSP, ° C. | 82.8 | 83.3 | 84.4 | 87.8 | 80.0 |
| Viscosity (162.8° C.), cP | 9,504 | 15,488 | 9,841 | 11,938 | 13,860 |
| Green Creep, mm | 0 | 4-5 | 3-4 | 5-40 | 5-40 |
| Initial Retention, % | 94.6 (1.0) | 96.7 (1.2) | 95.3 (1.5) | 92.7 (3.1) | 88.6 (3.8) |
| (Standard Deviation) | | | | | |
| One-Week Retention, % | 93.6 (1.9) | 95.0 (1.4) | 90.4 (2.0) | 65.5 (11.2) | 79.6 (9.3) |
| (Standard Deviation) | | | | | |
| Two-Week Retention, % | 94.6 (2.5) | 89.5 (3.3) | 81.3 (2.1) | 75.4 (4.8) | 71.3 (17.4) |
| (Standard Deviation) | | | | | |
| Four-Week Retention, % | 93.0 (1.7) | 86.6 (3.7) | 86.5 (3.0) | 59.7 (11.2) | 68.8 (14.4) |
| (Standard Deviation) | | | | | |

For demanding elastic attachment applications, four-week aged creep retention values above 80% are typically required. As shown by the data, examples 1 to 3 prepared using tackifier levels above 35 wt % afford hot-melt adhesives excellent long-term creep retention (average values after full aging greater than 85% with relatively low standard deviation values). Conversely, the comparative examples, CE1 and CE2, produced using tackifying agent at charges less than 30 wt %, provide aged creep values below 70%. Adhesives displaying aged performance of CE1 and CE2 are not suitable for many applications at the add-on level employed.

Table 4 below provides further examples of the inventive strategy employing high tackifier formulations. As shown, replacing L-MODU 5600 with a poly(propylene-co-ethylene) material (Vistamaxx 6202 in Example 4), or a higher molecular weight, low tacticity, propylene homopolymer (L-MODU 5901 in Example 5) provides adhesives with acceptable aged creep performance. The performance is preserved moving to alternate APAO systems or lower RBSP tackifying agents as shown by examples 6 and 7.

TABLE 4
Further Examples of High Creep Performance Adhesives

| Example | 4 | 5^{b} | 6 | 7 |
|---|---|---|---|---|
| Escorez 5400, wt % | — | — | — | 54.5 |
| Sukorez SU-210, wt % | 54.5 | 54.5 | 54.5 | — |
| Rextac 2330, wt % | — | — | 25.0 | — |
| Vestoplast 508 | — | — | — | 25.0 |
| Vestoplast EP NC 702, wt % | 25.0 | 25.0 | — | — |
| L-MODU S600, wt % | — | — | — | — |
| L-MODU S901, wt % | — | 20.0 | 20.0 | 20.0 |
| Vistamaxx 6202, wt % | 20.0 | — | — | — |
| Irganox 1010, wt % | 0.5 | 0.5 | 0.5 | 0.5 |
| RBSP, ° C. | 84.4 | 88.3 | 94.4 | 83.9 |
| Viscosity (162.8° C.), cP | 33,836^{a} | 13,801 | 33,484 | 38,852 |
| Green Creep, mm | 0 | 0 | 0 | 0 |
| Initial Retention, % | 97.0 (0.9) | 93.9 (2.9) | 90.4 (11.7) | 91.9 (7.0) |
| (Standard Deviation) | | | | |
| One-Week Retention, % | 93.2 (1.9) | 96.8 (1.2) | 95.4 (4.5) | 96.2 (2.8) |
| (Standard Deviation) | | | | |
| Two-Week Retention, % | 85.3 (3.6) | 71.3 (14.4) | 95.1 (2.6) | 95.3 (1.6) |
| (Standard Deviation) | | | | |
| Four-Week Retention, % | 93.2 (1.2) | 85.3 (2.7) | 94.7 (4.7) | 96.0 (0.7) |
| (Standard Deviation) | | | | |

NOTES:
^{a}Viscosity measured at 176.7° C.
^{b}Add-on decreased to 25 mg adhesive/m strand; 15 gsm FQN used for both substrates

It can be further advantageous for an elastic component adhesive to run under a range of process conditions and application methods. Example 5A, which duplicates the formulation and run conditions of Example 5 in the absence of employing nip-roller compression, demonstrates that creep retention performance is maintained even using a non-restrictive “S-wrap” line configuration. Example 5B shows performance data for the Example 5 formulation applied using Nordson Surewrap® indirect spray applicator. Here the excellent creep resistance of Example 5 is preserved demonstrating that the inventive formulations can also be run in spray applications without negatively impacting creep performance.

TABLE 5
Creep Performance of Ex5 at Further Process Parameters

| Example | 5A | 5B |
|---|---|---|
| Application Nozzle | Allegro ™ | Surewrap ® |
| Compression, psi | 0 | 40 |
| Green Creep, mm | 0 | 1-2 |
| Initial Retention, % | 96.4 (1.3) | 93.0 (2.1) |
| (Standard Deviation) | | |
| One-Week Retention, % | 95.2 (2.0) | 95.5 (1.5) |
| (Standard Deviation) | | |
| Two-Week Retention, % | 92.2 (4.0) | 93.2 (2.4) |
| (Standard Deviation) | | |
| Four-Week Retention, % | 94.8 (3.0) | 94.7 (1.5) |
| (Standard Deviation) | | |

### Examples 8-12 Testing in Construction Adhesive Applications

The adhesives of these examples shown in Table 6 below were made using a traditional overhead mixer at 176.7° C. First, the oil (Nyflex 222B), tackifier (Escorez or Sukorez), and antioxidants (Irgafos and Irganox) were heated to the desired temperature and the mixture was stirred for homogeneity. The L-MODU S400 was added first, then the Vestoplast 508 was added. After all polymer was dissolved and the mix appeared homogenous, the viscosity was tested. If not specified, the amounts of constituents are in weight percent.

TABLE 6

| | Ex. 8 | Ex. 9 | Ex. 10 |
|---|---|---|---|
| NYFLEX 222B | 16.50 | 12.50 | 12.50 |
| ESCOREZ 5615 | 40.00 | | |
| SUKOREZ SU-210 | | 40.00 | |
| SUKOREZ SU-100 | | | 40.00 |
| IRAGAFOS 169 | 0.35 | 0.35 | 0.35 |
| IRGANOX 1010 | 0.15 | 0.15 | 0.15 |
| L-MODU S400 | 19.00 | 23.00 | 23.00 |
| VESTOPLAST 508 | 24.00 | 24.00 | 24.00 |
| | 100.00 | 100.00 | 100.00 |
| Softening ° C. | 73.9 | 73.7 | 70.0 |
| Viscosity at 148.9° C., cP | 4,000 | 3,795 | 5,300 |

The adhesives were coated in a continuous fashion to a non-breathable film (DH284 by Clopay) using a two-inch Universal™ Signature™ spray applicator (Nordson Corporation). The application temperature was 148.9° C. with a line speed of 900 ft/min, an open time of 0.25 sec, and a 40 psi compression. The coated primary substrate was joined with a secondary substrate that was a 15 grams per square meter nonwoven (available from First Quality). In order to determine the adhesive peel performance, the laminates were allowed to age for 24 hours before being pulled apart by an Instron tensile tester at a rate of 12 inches per minute in a climate controlled room, which was maintained at a constant 23.9° C. and 50% relative humidity. The peel force was measured in grams-force, and the peel value was calculated by determining the average peel strength after eliminating the first and last five percent of the sample length to reduce variability from starting and stopping the test. This test was performed using an add-on level of two grams per square meter.

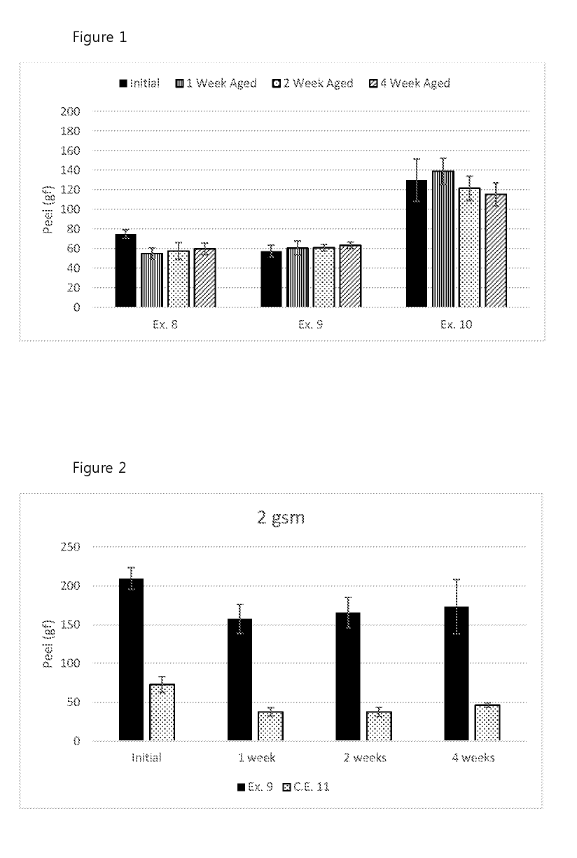

FIG. 1 shows peel performance at 1 gram per square meter. The results of FIG. 1 show that all formulations have acceptable peel performance at only 1 gram per square meter. It is preferred to have a peel strength at or above 100 grams, so the adhesive of Example 10 is particularly preferred.

For comparative purposes, the adhesive of Example 10 was tested against a comparative example (CE 11). Provided in Table 7 is the formulation of CE11, which uses Affinity GA 1900, a propylene-ethylene copolymer:

TABLE 7

| Raw Material | CE11 |
|---|---|
| NYFLEX 222B | 15.70 |
| SUKOREZ SU-100 | 53.50 |
| IRGAFOS 168 | 0.30 |
| IRGANOX 1010 | 0.50 |
| AFFINITY GA 1900 | 28 |
| BAS 450 SD | 2.00 |
| | 100.00 |

FIG. 2 shows the improved peel strength at 2 grams per square meter add-on with Signature applicator nozzles of the inventive polyolefin (Ex 10) compared to a non-inventive formulation (CE 11). The adhesive of the present invention demonstrates peel strength values generally over three times the peel strength of the non-inventive formulation.

The minimum shear strength of the adhesives was also tested. A minimum time of twenty minutes was determined by slot coating a one-inch pattern at fifteen grams per square meter with a one second open time to a heavy substrate. The substrate used as both the primary and secondary substrate was a blue SMS medical drape. One-inch strips of laminate were cut so that the primary substrate was hung in an oven at 38.9° C., and the secondary substrate had a 250 g weight hung from it. Once the laminate falls apart cohesively, the weight stops the timer. Preferably, the inventive formulation has a shear strength of at least 100 minutes compared to other polyolefin-based formulation having a value of less than one minute.

A formulation, identified as Example 12 in Table 8 below, was prepared to be identical to Example 10 except that Vistamaxx 6202 was used in place of L-MODU 400:

TABLE 8

| Example | 12 |
|---|---|
| Nyflex 222B | 12.5 |
| Sukorez SU-100 | 40.0 |
| Vestoplast 508 | 24.0 |
| Vistamaxx 6202 | 23.0 |
| Irgafos 168 | 0.35 |
| Irganox 1010 | 0.15 |
| RBSP, ° C. | 94.9 |
| Viscosity (162.8° C.), cP | 69,750 |

The same peel performance test was conducted as described above to add-on weights of 1.0, 2.0, and 4.2 grams per square meter. Provided below in Table 9 are the initial values and one-week aged values for both Examples 10 and 12 at the three add-on weights.

TABLE 9

| Add-on | Example 10 | | Example 12 | |
|---|---|---|---|---|
| (gsm) | AVG Peel (gf) | StDev | AVG Peel (gf) | StDev |
| 1.0 | 130.2 | 21.62 | 110.91 | 11.56 |
| 2.0 | 289.02 | 16.19 | 233.81 | 17.73 |
| 4.2 | 978.14 | 125.02 | 560.35 | 39.34 |

1 Week Aged at 54.5° C.

| | | | | |
|---|---|---|---|---|
| 1.0 | 139.21 | 13.39 | 146.89 | 33.87 |
| 2.0 | 392.01 | 37.06 | 340.03 | 36.45 |
| 4.2 | 1080.11 | 85.27 | 1045.96 | 128.2 |

As shown from Table 9, both Examples 10 and 12 performs well in both the initial and one-week aged peel performance test. In general, Example 10 performed better than Example 12 across all three add-on weights tested in the initial peel performed tests, but the degree of improvement was less noticeable in the one-week aged values. In fact, Example 12 performed better (although within standard deviation) in the one-week aged values for an add-on weight of one gram per square meter.

Table 10 below shows two additional examples of the invention, Examples 13 and 14, which differ primarily in the amount of plasticizer, KN4010, which is a naphthenic oil available from Karamay Petrochem Co. In these examples, the add-on weight was 35 mg/m strand, and a line speed of 900 ft/min was used. The substrates used for the creep test to assess suitability as an elastic component adhesive were breathable BR-134 and 15 gsm FQN NW (as defined above). The peel conditions were at 1 gsm on non-breathable DH-284 substrate to 15 gsm FQN NW at 900 ft/min line speed (as defined above).

TABLE 10

| Example | EX 13 | EX 14 |
|---|---|---|
| KN4010, wt % | 10.0 | 15.0 |
| Irganox 1010, wt % | 0.5 | 0.5 |
| Sukorez Su-210, wt % | 51.6 | 48.7 |
| L-MODU S901, wt % | 14.2 | 13.4 |
| Vestoplast EP NC 702, wt % | 23.7 | 22.4 |
| RBSP, ° C. | 81.9 | 79.1 |
| Viscosity (162.8° C.), cP | 6,500 | 4,185 |
| Green Creep, mm | 0-1 | 0-1 |
| Initial Retention, % | 93.9 | 92.0 |
| (Standard Deviation) | 0.3 | 1.4 |
| One-Week Retention, % | 93.7 | 85.5 |
| (Standard Deviation) | 0.9 | 2.2 |
| Two-Week Retention, % | 92.4 | 83.4 |
| (Standard Deviation) | 2.2 | 1.6 |
| Four-Week Retention, % | 90.7 | 83.4 |
| (Standard Deviation) | 0.8 | 3.1 |
| Initial Peel, gf | 58.5 | 120.8 |
| (Standard Deviation) | 10.7 | 19.2 |
| One-Week Peel, gf | 120.5 | 160.1 |
| (Standard Deviation) | 15.0 | 20.9 |

As can be seen, Example 13 does not meet the most stringent initial peel performance criterion of at least 100 gf peel strength both initially and after aging. This adhesive may be appropriate for in certain applications, such as in elastic applications. Example 14 exhibits both creep retention above 80% both initially and at all aged times tested and 1 gsm peel above 100 gf both initially and after aging for one week.

Where a range of values is provided, it is understood that each intervening value, and any combination or sub-combination of intervening values, between the upper and lower limit of that range and any other stated or intervening value in that stated range, is encompassed within the range of values recited. In addition, the invention includes a range of a constituent which is the lower limit of a first range and an upper limit of a second range of that constituent.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number, and thus will typically refer to a number or value that is 10% below or above the specifically recited number or value.

Unless defined otherwise, all technical and scientific terms used herein have the same meanings as commonly understood by one of ordinary skill in the art to which this invention belongs. All publications and patents specifically mentioned herein are incorporated by reference in their entirety for all purposes including describing and disclosing the chemicals, instruments, statistical analyses and methodologies which are reported in the publications which might be used in connection with the invention. Although illustrated and described herein with reference to certain specific embodiments, the present invention is nevertheless not intended to be limited to the details shown. Rather, various modifications may be made in the details within the scope and range of equivalents of the claims and without departing from the spirit of the invention.

What is claimed:

1. A hot melt adhesive composition comprising:
(a) about 2% to about 50% by weight of a first polymer component having a low melting point and selected from the group consisting of a polypropylene homopolymer and a copolymer of propylene and ethylene and mixtures thereof;
(b) about 2% to about 50% by weight of a second polymer component comprising an amorphous polyolefin; and
(c) about 30% to about 75% by weight of a tackifying resin having a Ring & Ball softening point of at least about 80° C. and up to about 140° C.,
wherein the viscosity of the composition is equal to or less than about 80,000 cP at 180° C.

2. The composition of claim 1, wherein the tackifying resin is present in an amount of about 32% to about 73% by weight.

3. The composition of claim 1, wherein the tackifying resin is present in an amount of about 32% to 55%.

4. The composition of claim 1, wherein the weight ratio of the first polymer component to the second polymer component varies from about 1:3 to about 5:4.

5. The composition of claim 1, wherein the tackifying resin has a RBSP of at least about 85° C. and to about 135° C.

6. The composition of claim 1, wherein the tackifier is selected from the group consisting of aliphatic and cycloaliphatic hydrocarbon resins and their hydrogenated derivatives, hydrogenated aromatic hydrocarbon resins, aromatically modified aliphatic or cycloaliphatic resins and their hydrogenated derivatives, polyterpene and styrenated polyterpene resins and mixtures thereof.

7. The composition of claim 1, wherein the tackifier is selected from the group consisting of a C-5 aliphatic hydrocarbon resin, a hydrogenated C-5 resin, a hydrogenated C-9 resin, a hydrogenated DCPD resin and an aromatic-modified DCPD resin.

8. The composition of claim 1, further comprising a plasticizer in an amount of about 0.1% to about 20% by weight.

9. The composition of claim 8, wherein the plasticizer is selected from the group consisting of mineral oil and liquid polybutene.

10. The composition of claim 9, wherein the plasticizer is mineral oil and the mineral oil has less than 30% aromatic carbon atoms.

11. The composition of claim 1, wherein the adhesive composition further comprises a wax in the amount up to 20% by weight.

12. The composition of claim 11, wherein said wax is selected from the group consisting of petroleum waxes, low molecular weight polyethylene and polypropylene, synthetic waxes and polyolefin waxes and mixtures thereof.

13. The composition of claim 11, wherein said wax is a low molecular weight polyethylene having a number average molecular weight of about 400 to about 6,000 g/mol.

14. The composition of claim 1 further comprising at least one of a stabilizer or an antioxidant.

15. The composition of claim 14, wherein said at least one stabilizer or antioxidant is an antioxidant and said antioxidant is a hindered phenol compound.

16. The composition of claim 1 further comprising a filler in the amount up to 60% by weight.

17. The composition of claim 16, wherein said filler is selected from the group consisting of talc, calcium carbonate, clay, silica, mica, wollastonite, feldspar, aluminum silicate, alumina, hydrated alumina, glass microsphere, ceramic microsphere, thermoplastic microsphere, baryte and wood flour and mixtures thereof.

18. The composition of claim 1 further comprising a third polymer component.

19. The composition of claim 18, wherein said third polymer component is selected from the group consisting of EVA, PE, LDPE, LLDPE, PB, and a styrenic block copolymer and mixtures thereof.

20. The composition of claim 19, wherein said third polymer component is said styrenic block copolymer and said styrenic block copolymer is selected from the group consisting of SIS, SI, SBS, SB, SIBS, SEB, SEBS, SEP, SEPS, SBBS, SEEPS and mixtures thereof.

21. The composition of claim 1, wherein said first polymer component has a DSC melting point of less than 100° C.

22. The composition of claim 1, wherein said first polymer has a modulus defined by having a value of at least 20% elongation at break according to ASTM D638.

23. The composition of claim 1, wherein the first polymer component is a polypropylene homopolymer and has a DSC melting point of less than 100° C.

24. The composition of claim 1, wherein said second polymer component comprises a polyalphaolefin.

25. The composition of claim 24, wherein said polyalphaolefin comprises a poly(l-butene-co-propylene) polymer.

26. A method of making a laminate comprising the steps of:
applying the hot melt adhesive composition of claim 1 in a molten state to a first substrate; and
mating a secondary substrate to the first substrate by contacting the secondary substrate with the adhesive composition.

27. The method of claim 26, wherein the adhesive is applied to the first substrate using a direct contact method of hot melt application.

28. The method of claim 26, wherein the adhesive is applied to the first substrate using a non-contact method of hot melt application.

29. The method of claim 26, wherein the first substrate is an elastic strand.

30. The method of claim 26, wherein the first substrate is a nonwoven fabric.

31. The method of claim 29, wherein the secondary substrate is a nonwoven fabric wrapped around the elastic strand.

32. The method of claim 29, wherein the secondary substrate is a polyethylene film and a tertiary substrate is a nonwoven fabric.

33. A laminate made by the method of claim 26 used as an elastic leg cuff, a standing leg cuff or an elastic side panel in a disposable article.

34. A disposable article comprising a composition of claim 1 and at least one substrate.

35. The method of claim 26, wherein the first substrate is a polyolefin film and the second substrate is a nonwoven material.

36. The composition of claim 1, wherein the first polymer component is present in an amount of about 5% to about 35% by weight of the composition.

37. The composition of claim 1, wherein the first polymer component is present in an amount of about 15% to about 38% by weight of the composition.

38. The composition of claim 1, wherein the second polymer component is present in an amount of about 10% to about 45% by weight of the composition.

39. The composition of claim 1, wherein the second polymer component is present in an amount of about 10% to about 40% by weight of the composition.

40. The composition of claim 1, wherein the weight ratio of total polymer to tackifying resin varies from about 3:7 to about 7:3.

41. The composition of claim 1, wherein the first polymer component is a propylene-co-ethylene polymer and the second polymer component is a APAO.

42. The composition of claim 8, wherein the first polymer component is present in an amount of from about 5% to about 30% by weight, the second polymer component is present in an amount of from about 15% to about 40%, the tackifying resin is present in an amount of from about 30% to about 70% by weight, and the plasticizer is present in the adhesive composition in amounts of from about 5% to about 20% by weight.

43. The composition of claim 42, wherein the weight ratio of the first polymer component to the second polymer component varies from about 1:5 to about 1:1 and the weight ratio of total polymer:tackifying resin varies from about 1:3 to about 3:2.

44. A hot melt adhesive composition comprising:
a first polymer component having a low melting point and selected from the group consisting of a polypropylene homopolymer and a copolymer of propylene and ethylene and mixtures thereof;
a second polymer component comprising an amorphous polyolefin; and
a tackifying resin having a Ring & Ball softening point of at least about 80° C. and up to about 140° C.,
wherein the viscosity of the composition is equal to or less than about 80,000 cP at 180° C. and the first polymer component, the second polymer component, and the tackifying resin are present in amounts effective to provide a hot melt adhesive composition which: (1) has a peel strength of at or above 100 grams-force at 1 gram per square meter both initially and after aging for 1 week and (2) a creep retention of at least 80% both initially and after aging for 1, 2, and 4 weeks.

45. A method for using a dually functional adhesive comprising the steps of:
melting a single batch of an adhesive to form a molten adhesive;
dividing the molten adhesive into a first portion and a second portion;
directing the first portion to a first region of a plant and applying the adhesive at the first region to at least one of a first substrate or an elastic component to provide a first adhesive-bearing surface;
attaching the other of the first substrate or the elastic component to the first adhesive-bearing surface;
directing the second portion to a second region of the plant and applying the adhesive at the second region to at least one of a second substrate or a nonwoven layer to provide a second adhesive-bearing surface; and
attaching the other of the second substrate or the nonwoven layer to the second adhesive-bearing surface,
wherein the adhesive is effective to provide: (1) a peel strength of at or above 100 grams-force at 1 gram per square meter both initially and after aging for 1 week and (2) a creep retention of at least 80% both initially and after aging for 1, 2, and 4 weeks.

46. The method of claim 45, further comprising, before applying the adhesive at the second section to the second substrate, adding a plasticizer to the second portion of the adhesive.
